(19) United States
(12) Patent Application Publication
(10) Pub. No.: US 2026/0308643 A1
(43) Pub. Date: Oct. 8, 2026

(54) METHOD FOR PRODUCING BIOETHANOL AND DEVICE FOR PRODUCING BIOETHANOL

(71) Applicant: ARKHE Co., Ltd., Saitama (JP)

(72) Inventors: Shinobu YOSHIKAWA, Saitama (JP); Yoshiya IZUMI, Fukuoka (JP)

(73) Assignee: ARKHE Co., Ltd., Saitama (JP)

(21) Appl. No.: 19/640,847

(22) Filed: Apr. 7, 2026

(30) Foreign Application Priority Data
Apr. 7, 2025 (JP) 2025-063354

(51) Int. Cl. C12P 7/06; B01D 3/14; B01D 61/02; B01D 61/08; B01D 61/14; B01D 61/18; B01D 61/58; B01D 69/02; B01D 69/14; B01D 71/02; B01D 71/56; C12M 1/00

(52) U.S. Cl. CPC C12P 7/06; B01D 3/145; B01D 61/08; B01D 61/18; B01D 61/58; B01D 69/02; B01D 69/144; B01D 71/02; B01D 71/56; C12M 21/12; C12M 47/10; B01D 61/025; B01D 61/147; B01D 61/149; B01D 2325/20

(57) ABSTRACT

A method for producing second-generation bioethanol with a reduced production cost. The method for producing bioethanol, which is a method for producing bioethanol from cellulose, includes: in order, a saccharification step of adding an enzyme to the cellulose for saccharification to obtain a sugar liquid; a step of obtaining a concentrated sugar liquid by concentrating the obtained sugar liquid; a fermentation step of adding yeast to the concentrated sugar liquid for fermentation to obtain hydrous bioethanol; and a concentration step of concentrating the hydrous bioethanol to obtain bioethanol. The method further includes at least one of a step of collecting the enzyme using a ceramic membrane after the saccharification step and a step of collecting unreacted yeast using a ceramic membrane after the fermentation step, and/or in the step of obtaining the concentrated sugar liquid, the sugar liquid is concentrated using a protein-supported polyamide RO membrane.

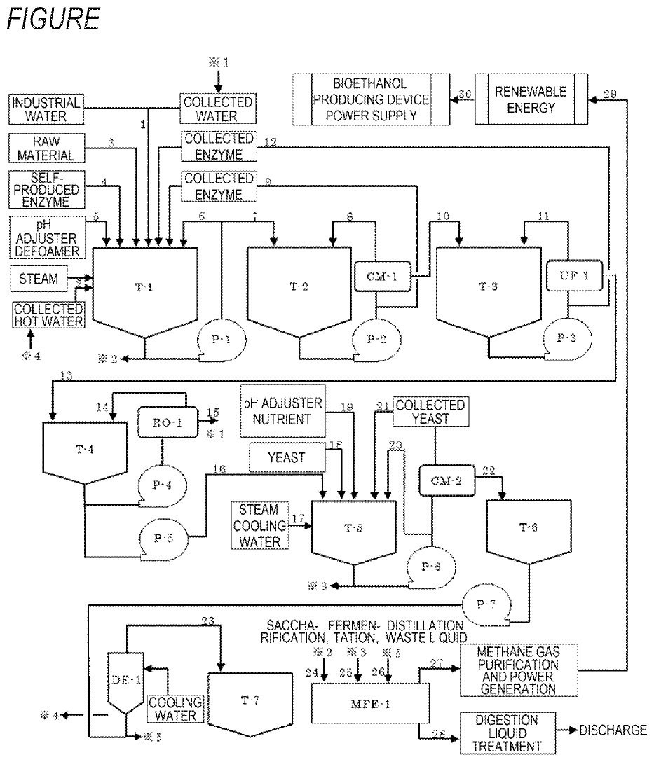

### TECHNICAL FIELD

The present invention relates to a method for producing bioethanol and a device for producing bioethanol.

### BACKGROUND ART

In recent years, the concentration of carbon dioxide (CO₂) in the air has increased due to consumption of fossil fuel, and global warming due to the increase has become a serious problem.

Therefore, bioethanol has been studied and promoted as eco-fuel having a CO₂ emission amount in a life cycle smaller than that of fossil fuel.

A raw material for bioethanol is mainly a plant-derived raw material such as a starchy raw material such as corn, a sweet potato, barley, or tapioca, or a carbohydrate raw material such as sugarcane. High purity ethanol is obtained by fermenting sugar contained in the plant-derived raw material using microorganisms and then performing distillation.

On the other hand, since the plant-derived raw material is also an edible raw material, when a demand for eco-fuel increases and a consumption amount thereof increases, an amount of food supplied decreases, which may lead to a rise in food prices.

Therefore, in recent years, development of second-generation bioethanol using a cellulose-based raw material such as a woody material or a herbaceous plant, which is a non-edible raw material, has been advanced.

Since the cellulose-based raw material does not easily accept the action of an enzyme or yeast traditionally used for obtaining bioethanol, a production technique combining a pretreatment, saccharification, fermentation, concentration, and the like is required. Therefore, the production cost is higher than that of bioethanol in the related art.

Therefore, in order to widely spread second-generation bioethanol, technical development for cost reduction is expected.

Since there are various techniques for producing second-generation bioethanol, there are various methods for implementing cost reduction.

For example, Patent Literature 1 discloses a method for producing bioethanol that can increase a concentration of hydrous bioethanol and reducing a distillation load without requiring special equipment.

Patent Literature 2 discloses a fermentation method for promoting progress of an enzyme decomposition reaction when cellulose is decomposed and saccharified by an enzyme.

### CITATION LIST

### Patent Literature

Patent Literature 1: JP2019-106954A

Patent Literature 2: JP2002-186938A

### SUMMARY OF INVENTION

### Technical Problem

On the other hand, the present inventors have found a new approach to provide a method for producing second-generation bioethanol with a reduced production cost.

That is, an object of the present invention is to provide a method for producing second-generation bioethanol with a reduced production cost. Another object of the present invention is to provide a device for producing second-generation bioethanol with a reduced production cost as described above.

### Solution to Problem

In view of the above, the present inventors have conducted intensive studies, and have conceived a method for solving the above problems using a membrane separation technique. Specifically, the present inventors have conceived that by suitably collecting an enzyme and unreacted yeast by using a ceramic membrane, the enzyme and the unreacted yeast can be used again, and a sugar liquid can be efficiently concentrated with low energy by using a protein-supported polyamide RO membrane, and have completed the present invention.

The gist of an embodiment of the present invention will be described below.
[1] A method for producing bioethanol, which is a method for producing bioethanol from cellulose, the method including: in order,
a saccharification step of adding an enzyme to the cellulose for saccharification to obtain a sugar liquid;
a step of obtaining a concentrated sugar liquid by concentrating the obtained sugar liquid;
a fermentation step of adding yeast to the concentrated sugar liquid for fermentation to obtain hydrous bioethanol; and
a concentration step of concentrating the hydrous bioethanol to obtain bioethanol, in which
the method further includes at least one step of an enzyme collection step performed after the saccharification step and an unreacted yeast collection step performed after the fermentation step,
the enzyme collection step includes a step of collecting the enzyme using a ceramic membrane, and
in the unreacted yeast collection step, a ceramic membrane is used.
[2] A method for producing bioethanol, which is a method for producing bioethanol from cellulose, the method including: in order,
a saccharification step of adding an enzyme to the cellulose for saccharification to obtain a sugar liquid;
a step of obtaining a concentrated sugar liquid by concentrating the obtained sugar liquid;
a fermentation step of adding yeast to the concentrated sugar liquid for fermentation to obtain hydrous bioethanol; and
a concentration step of concentrating the hydrous bioethanol to obtain bioethanol, in which
in the step of obtaining the concentrated sugar liquid, the sugar liquid is concentrated using a protein-supported polyamide RO membrane.
[3] The method for producing bioethanol according to the above 1, in which
the enzyme collection step further includes a step of collecting the enzyme using a UF membrane after the step of collecting the enzyme using the ceramic membrane.
[4] The method for producing bioethanol according to the above [3], in which
a UF membrane having a molecular weight cut-off of MW-3000 to MW-7000 is used as the UF membrane.
[5] The method for producing bioethanol according to the above [1], [3], or [4], in which
in the enzyme collection step, undecomposed cellulose is also collected together with the enzyme.
[6] The method for producing bioethanol according to any one of the above [1] and [3] to [5], in which
the enzyme collection step and the unreacted yeast collection step are both performed.
[7] The method for producing bioethanol according to any one of the above [1] and [3] to [6], in which
in the step of obtaining the concentrated sugar liquid, the sugar liquid is concentrated using an RO membrane.
[8] The method for producing bioethanol according to the above [7], in which
a protein-supported polyamide RO membrane is used as the RO membrane.
[9] The method for producing bioethanol according to any one of the above [1] and [3] to [8], in which
in at least one of the enzyme collection step and the unreacted yeast collection step, a ceramic membrane having filtration accuracy of 1.2 μm to 1.6 μm is used as the ceramic membrane.
[10] The method for producing bioethanol according to any one of the above [1] to [9], in which
in the concentration step, the hydrous bioethanol is concentrated by distillation.
[11] The method for producing bioethanol according to any one of the above [1] to [10], further including:
a step of collecting a protein-containing waste liquid after one or more steps of the saccharification step, the fermentation step, and the concentration step.
[12] The method for producing bioethanol according to the above [11], further including:
a step of producing and collecting methane gas using the collected protein-containing waste liquid.
[13] A device for producing bioethanol, which is a device for producing bioethanol from cellulose, the device including:
a saccharification tank configured to allow an enzyme to be added to the cellulose for saccharification to obtain a sugar liquid;
a sugar liquid tank configured to allow a concentrated sugar liquid obtained by concentrating the obtained sugar liquid to be supplied;
a fermentation tank configured to allow yeast to be added to the concentrated sugar liquid for fermentation to obtain hydrous bioethanol; and
a bioethanol tank configured to allow bioethanol obtained by concentrating the hydrous bioethanol to be supplied, in which
at least one of a ceramic membrane for separating the enzyme provided between the sugar liquid tank and the fermentation tank and a ceramic membrane for separating unreacted yeast provided between the fermentation tank and the bioethanol tank is further provided.
[14] A device for producing bioethanol, which is a device for producing bioethanol from cellulose, the device including:
a saccharification tank configured to allow an enzyme to be added to the cellulose for saccharification to obtain a sugar liquid;
a sugar liquid tank configured to allow a concentrated sugar liquid obtained by concentrating the obtained sugar liquid to be supplied;
a fermentation tank configured to allow yeast to be added to the concentrated sugar liquid for fermentation to obtain hydrous bioethanol; and
a bioethanol tank configured to allow bioethanol obtained by concentrating the hydrous bioethanol to be supplied, in which
a protein-supported polyamide RO membrane for concentrating the sugar liquid is provided between the sugar liquid tank and the fermentation tank.
[15] The device for producing bioethanol according to the above [13], in which
a protein-supported polyamide RO membrane for concentrating the sugar liquid is provided between the sugar liquid tank and the fermentation tank.

### Advantageous Effects of Invention

According to the present embodiment, second-generation bioethanol can be produced at a low production cost.

More specifically, by using the ceramic membrane, the enzyme and unreacted yeast can be suitably collected, and the collected enzyme and unreacted yeast can be reused. An amount of saccharification residue waste liquid and an amount of fermentation residue waste liquid can also be reduced. Further, by using the protein-supported polyamide RO membrane, the concentration of the sugar liquid can be efficiently implemented with very low energy. As a result, the production cost of bioethanol can be significantly lower than before.

### BRIEF DESCRIPTION OF DRAWINGS

FIGURE is a schematic configuration diagram illustrating an aspect of a device for producing bioethanol according to the present embodiment.

### DESCRIPTION OF EMBODIMENTS

Hereinafter, an embodiment of the present invention will be described. The present invention is not limited to the embodiment described below.

In the present specification, “to” indicating a numerical range is used to include numerical values written before and after it as a lower limit value and an upper limit value.

### Method for Producing Bioethanol

An aspect of a method for producing bioethanol according to the present embodiment includes the following steps in order.

A saccharification step of adding an enzyme to the cellulose for saccharification to obtain a sugar liquid,
a step of obtaining a concentrated sugar liquid by concentrating the obtained sugar liquid,
a fermentation step of adding yeast to the concentrated sugar liquid for fermentation to obtain hydrous bioethanol, and
a concentration step of concentrating the hydrous bioethanol to obtain bioethanol.

In addition to the above, the producing method according to the present embodiment preferably further includes an enzyme collection step after the saccharification step, preferably further includes an unreacted yeast collection step after the fermentation step, and more preferably further includes both.

Here, the enzyme collection step preferably includes a step of collecting the enzyme using a ceramic membrane. In the unreacted yeast collection step, the ceramic membrane is preferably used.

In addition to the above, in the producing method according to the present embodiment, in the step of obtaining the concentrated sugar liquid, it is preferable to concentrate the sugar liquid using a reverse osmosis membrane (an RO membrane), and it is more preferable that the RO membrane is a protein-supported polyamide RO membrane.

Another aspect of the method for producing bioethanol according to the present embodiment includes the following steps in order.

A saccharification step of adding an enzyme to the cellulose for saccharification to obtain a sugar liquid,
a step of obtaining a concentrated sugar liquid by concentrating the obtained sugar liquid,
a fermentation step of adding yeast to the concentrated sugar liquid for fermentation to obtain hydrous bioethanol, and
a concentration step of concentrating the hydrous bioethanol to obtain bioethanol.

In the step of obtaining the concentrated sugar liquid, it is preferable to concentrate the sugar liquid using a protein-supported polyamide RO membrane.

In the producing method according to the present embodiment, it is preferable to use a ceramic membrane in the enzyme collection step after the saccharification step and the unreacted yeast collection step after the fermentation step. Accordingly, the enzyme and the unreacted yeast can be suitably collected.

As a result, the enzyme and unreacted yeast can be used again in the saccharification step and the fermentation step, an amount of enzyme to be used and an amount of yeast to be used can be reduced, and further, an amount of saccharification residue waste liquid and an amount of fermentation residue waste liquid can also be reduced. From such a viewpoint, according to the present embodiment, bioethanol can be produced at a lower production cost.

In the producing method according to the present embodiment, in the step of obtaining the concentrated sugar liquid, it is preferable to concentrate the sugar liquid using an RO membrane. It was conceived that the concentration can be performed at low energy by using the RO membrane, and can be performed at lower energy by using the protein-supported polyamide RO membrane. Also from such a viewpoint, according to the present embodiment, bioethanol can be produced at a lower production cost.

Hereinafter, the two aspects will be collectively described for each step with reference to FIGURE.

The embodiment described in the drawing is schematically illustrated to clearly describe the present invention, and does not necessarily accurately represent the size or scale of an actual device or the like. The producing method according to the present embodiment is not limited to the embodiment described in the drawing.

### Saccharification Step

The saccharification step in the present embodiment is a step of adding an enzyme to cellulose for saccharification to obtain a sugar liquid.

Specifically, for example, cellulose and an enzyme as raw materials are charged into a saccharification tank T-1 (arrows 3 and 4 in FIGURE).

At this time, in addition to the cellulose and the enzyme, other components used in the related art may be further charged into the saccharification tank T-1. Examples of the other components include water (industrial water), a pH adjuster, and defoamer steam (arrows 1, 2, and 5 in FIGURE).

In the present embodiment, as the cellulose, for example, lignocellulose is suitably used, and those extracted from raw material pulp, wood, and other plants can be used. An extraction source for cellulose may be one kind or two or more kinds.

The raw material pulp is preferably unbleached softwood kraft pulp (NUKP) or bleached hardwood kraft pulp (LBKP) from the viewpoint of sustainable raw material supply.

The cellulose may be subjected to a pretreatment as necessary in order to loosen binding of cellulose, hemicellulose, and lignin. Accordingly, saccharification and fermentation become more likely to occur.

As the pretreatment, a method known in the related art can be adopted, and examples thereof include steam explosion or hydrothermal treatment using water, a concentrated sulfuric acid method or a dilute sulfuric acid method using an acid, an ammonia freezing explosion method using an alkali, a new isotropic ammonia method, an alkaline cooking method or a lime method, and an acetic acid method or an ethanol method using an organic solvent. These may be performed by one method or by a combination of two or more methods.

On the other hand, in view of an increase in energy required for the pretreatment and an increase in chemical consumption, the pretreatment may not be performed from the viewpoint of a producing cost.

As the pretreatment, centrifugation may be performed, and more specifically, centrifugation using a decanter-type centrifuge may be performed. The centrifugation has no concern about an increase in energy as described above.

As the enzyme in the present embodiment, an enzyme known in the related art can be used, and for example, cellulase is suitable.

The enzyme may be self-produced or a commercially available enzyme may be used, but in the case of being self-produced, for example, an enzyme produced by a continuous culture device is preferably used.

Cellulose has a polymer structure in which sugars are bonded together, and when hydrolyzed, cellulose is saccharified to form a sugar liquid. The addition of the enzyme at this time promotes the hydrolysis.

When the enzyme is added, a temperature in the saccharification tank T-1 may be maintained at an enzyme activation temperature by using steam. When cellulase is used as the enzyme, the enzyme activation temperature is preferably maintained in a range of 50° C.±3° C.

For the steam, for example, a method of charging steam into an outer jacket of the saccharification tank T-1 can be adopted.

Saccharification reaction conditions are not particularly limited, and conditions known in the related art can be adopted, and for example, a reaction temperature when cellulase is used as the enzyme is preferably 50° C.±3° C.

In a saccharification reaction when pulp is used as a raw material of cellulose, when a solid content concentration of the raw material is 54%, an enzyme activity of about 30 FPU/g-pulp is required per 1 kg of raw material. In accordance with this, the enzyme is charged into the saccharification tank T-1. In the case of reusing the collected enzyme, an amount of new enzyme to be charged is determined in accordance with an amount of the enzyme to be reused.

In the saccharification step in the present embodiment, a saccharification rate of 70% or more is preferably maintained, and a saccharification rate of 80% or more is more preferably maintained.

In the case of reusing the collected enzyme, in the producing method according to the present embodiment, the collected enzyme can be continuously reused in five or more batches. Therefore, even when an amount of enzyme to be newly added in the subsequent saccharification step is as remarkably small as about 20%, the saccharification step can be operated without any problem, and the effect of reducing the amount of the enzyme used in the production method according to the present embodiment is very large.

In the saccharification step in the present embodiment, for example, a mixed liquid containing about 80 mass % of the sugar liquid having a Brix value of about 8% to 9% and about 20 mass % of undecomposed cellulose is produced.

The undecomposed cellulose may have a particle size of about 4 μm to 1,000 μm or about 100 μm to 1,000 μm.

### Enzyme Collection Step

The producing method according to the present embodiment preferably includes the enzyme collection step after the saccharification step and before concentrating the sugar liquid obtained in the saccharification step.

Solid-liquid separation may be performed before the enzyme collection step. The sugar liquid after the solid-liquid separation is supplied to a sugar liquid tank T-2.

When the sugar liquid in the sugar liquid tank T-2 is microfiltered through a ceramic membrane CM-1, the enzyme is separated from the sugar liquid, the enzyme can be collected, and the sugar liquid is simultaneously purified.

The purification means that the undecomposed cellulose in the sugar liquid obtained in the saccharification step is separated from the sugar liquid. That is, in the step of collecting the enzyme using the ceramic membrane in the present embodiment, it is preferable to collect the undecomposed cellulose together with the enzyme.

It is preferable that the enzyme collected as described above is returned as a collected enzyme to the saccharification tank T-1 again and subjected to the saccharification step (an arrow 9 in FIGURE). At this time, when the undecomposed cellulose is also returned to the saccharification tank T-1, the cellulose is subjected to the saccharification step again, and the saccharification rate of the cellulose can be increased.

As described above, in the enzyme collection step in the present embodiment, the enzyme can be easily and efficiently collected together with the undecomposed cellulose as necessary by using the ceramic membrane CM-1. At the same time, purification of the sugar liquid, collection of the enzyme or undecomposed cellulose, and return to the saccharification tank T-1 can be continuously performed.

By repeatedly using the enzyme through collection and return of the enzyme, the amount of enzyme to be used can be reduced, leading to a reduction in production cost. In addition, reducing an amount of the enzyme contained in the waste liquid is advantageous from the viewpoint of reducing the environmental load. While repeatedly using the enzyme, a part of a new enzyme may be charged into the saccharification tank T-1 to be used.

The ceramic membrane CM-1 in the present embodiment preferably has filtration accuracy of 1.2 μm to 1.6 μm. Here, the filtration accuracy is preferably 1.2 μm or more, more preferably 1.25 μm or more, and still more preferably 1.3 μm or more, and is preferably 1.6 μm or less, more preferably 1.55 μm or less, and still more preferably 1.5 μm or less.

The filtration using the ceramic membrane CM-1 may be performed, for example, with a sugar liquid temperature at 40° C. to 50° C., a filtrate Flux at 70 (±10%) LMH, a circulation flow rate Flux at 30 (±5%) times, and a circulation pressure at 0.6 MPa to 0.8 MPa (±5%). The present invention is not limited to these conditions.

By using the ceramic membrane for collecting the enzyme, physical friction of the undecomposed cellulose having high crystallinity occurs on the surface of the ceramic membrane, and the particle size of the undecomposed cellulose decreases. Then, the saccharification reaction between the collected enzyme taken into a circulating liquid separated by the filtration through the ceramic membrane and the fine crystalline undecomposed cellulose having a small particle size further proceeds, and as a result, a sugar liquid having a higher concentration with a Brix value of about 9% to 10% is obtained.

Such stable production of the sugar liquid having a high concentration leads to an increase in amount of hydrous bioethanol obtained in the fermentation step to be described later. As a result, the effect of reducing the production cost is also obtained from the viewpoint of increasing a yield of bioethanol finally obtained.

The enzyme collection step in the present embodiment preferably further includes a step of collecting the enzyme using an ultrafiltration membrane (UF membrane) UF-1 after the step of collecting the enzyme using the ceramic membrane CM-1. In this case, the sugar liquid filtered by the ceramic membrane CM-1 is transferred to a sugar liquid tank T-3 and then supplied to the UF membrane UF-1.

Specifically, by performing filtration using the UF membrane UF-1 on the sugar liquid transferred to the sugar liquid tank T-3 through the step of collecting the enzyme using the ceramic membrane CM-1, a low molecular enzyme contained in the sugar liquid, which cannot be collected by the filtration using the ceramic membrane CM-1, can be separated and collected.

By performing the filtration using the UF membrane UF-1, low molecular undecomposed cellulose contained in the sugar liquid, which cannot be collected by the filtration using the ceramic membrane CM-1, can also be separated and collected.

By separating the low molecular enzyme and the undecomposed cellulose by the filtration using the UF membrane UF-1, the sugar liquid is simultaneously filtered with higher precision.

As described above, in the step of collecting the enzyme in the present embodiment, it is more preferable to perform multi-stage solid-liquid separation using the ceramic membrane CM-1 and the UF membrane UF-1.

As the UF membrane UF-1, a UF membrane known in the related art can be used, and for example, a UF membrane having a molecular weight cut-off of MW-3000 to MW-7000 is preferably used. Here, from the viewpoint of the Flux performance of an ultralow-pressure aquaporin protein membrane to be used in the subsequent concentration step, the molecular weight cut-off is preferably MW-3000 or more, more preferably MW-3500 or more, still more preferably MW-4000 or more, and even more preferably MW-4500 or more. From the viewpoint of removing a low molecular organic compound, the molecular weight cut-off is preferably MW-7000 or less, more preferably MW-6500 or less, still more preferably Mw-6000 or less, and even more preferably MW-5500 or less.

Before the enzyme collection step in the present embodiment, that is, before the step of collecting the enzyme using the ceramic membrane, centrifugation or the like may be performed as necessary. Accordingly, the collection efficiency of the sugar liquid, the undecomposed cellulose, and the enzyme can be increased. Specifically, by dehydrating the sugar liquid, collecting long undecomposed cellulose and the enzyme contained in a substrate thereof, and then using the ceramic membrane, the undecomposed cellulose of the order of μm and the enzyme can be separated, collected, and purified in a short time with the ceramic membrane.

As the centrifugation, for example, a decanter-type centrifuge is preferred.

### Step of Obtaining Concentrated Sugar Liquid

The step of obtaining a concentrated sugar liquid in the present embodiment is a step of concentrating the sugar liquid obtained through the saccharification step or the subsequent enzyme collection step to obtain a concentrated sugar liquid.

The sugar liquid filtered by the ceramic membrane CM-1 and the UF membrane UF-1 is transferred to a sugar liquid tank T-4, and when concentrating such a sugar liquid, it is preferable to use a membrane having high water permeability and capable of favorably separating an organic substance and an inorganic substance, and for example, an RO membrane RO-1 is preferred. Among them, it is more preferable to concentrate the sugar liquid using the protein-supported polyamide RO membrane since the sugar liquid can be suitably concentrated with lower energy.

When the filtration using the ceramic membrane CM-1 and the UF membrane UF-1 is not performed on the sugar liquid in the sugar liquid tank T-2, the sugar liquid tanks T-3 and T-4 are not necessary, and the sugar liquid in the sugar liquid tank T-2 is directly subjected to the filtration by the RO membranes RO-1. When the filtration using the UF membrane UF-1 is not performed on the sugar liquid in the sugar liquid tank T-2, the sugar liquid tank T-4 is not necessary, and the sugar liquid in the sugar liquid tank T-3 is directly subjected to the filtration by the RO membrane RO-1.

As the protein-supported polyamide RO membrane, for example, a composite polyamide membrane (an aquaporin protein membrane) having disposed thereon a protein that localizes to a cell membrane (an integral membrane protein), which is called aquaporin (AQP) and forms a channel within the cell membrane to regulate a flow of water into and out of a cell is more preferred, and an ultralow-pressure aquaporin protein membrane is particularly preferred.

Here, a pressure in the ultralow-pressure means a pump discharge pressure applied when supplying the sugar liquid to the protein-supported polyamide RO membrane. Although the specific pressure varies depending on a composition and a concentration of the sugar liquid, the pressure can be determined with reference to an osmotic pressure. For example, the osmotic pressure of a 10% sugar liquid (glucose) is 222 mOsmol/kg, and an operating pressure can be set low with reference to this value.

The protein-supported polyamide RO membrane is a membrane incorporating the theory of an ideal bio-membrane. Specifically, the protein-supported polyamide RO membrane is a membrane that utilizes biomimetic technology by incorporating a layer of natural aquaporin proteins into a skin layer of the membrane. That is, the aquaporin proteins are water channels found in nature, present in all living cells, and function as ultra-efficient and ultra-selective high-speed channels for water molecules.

Therefore, by using the protein-supported polyamide RO membrane, the membrane functions as a reverse osmosis membrane having high water permeability while maintaining a rejection ratio, the power consumption required for concentration is reduced, and the effect of reducing CO₂ emissions is obtained.

For example, when the power consumption in the case of concentrating the sugar liquid using an evaporation device is 100%, the power consumption in the case of concentrating the sugar liquid using the protein-supported polyamide RO membrane can be about 40% to 60%, which is very advantageous.

A thickness of the protein-supported polyamide RO membrane in the present embodiment also varies depending on a membrane configuration.

When the protein-supported polyamide RO membrane is a composite membrane composed of three layers of a polyamide thin film, a microporous polysulfone, and a polyester in this order from the surface, a thickness of the polyamide thin film is preferably, for example, 0.05 μm to 0.5 μm, a thickness of the microporous polysulfone is preferably, for example, 20 μm to 60 μm, and a thickness of the polyester is preferably, for example, 90 μm to 150 μm.

A cell membrane may be present between the polyamide thin film and the microporous polysulfone, and a thickness of the cell membrane is, for example, preferably 3 nm to 5 nm, and more preferably 3.5 nm to 4.5 nm. The protein present in such a cell membrane is called aquaporin. Accordingly, the water molecules and the organic substance can be separated at a high removal rate at a high speed.

By using such a protein-supported polyamide RO membrane, the Flux can be maintained at 8 LMH to 12 LMH, and glucose can be separated from water with a removal rate of 99.2% to 99.7%.

In the step of obtaining the concentrated sugar liquid in the present embodiment, for example, at an operation pressure of 3.7(+3 ) MPa and a Flux of 9 LMH, the power consumption required to obtain 1 L of concentrated sugar liquid with a desalination ratio of 99.5% or more and a Brix value of about 20% or more is only about 0.01 kW, enabling highly efficient concentration with extremely low energy consumption.

This is much lower than the operation energy required for an evaporation device (MVR type) or a concentration step using a high-pressure reverse osmosis membrane.

Water separated when obtaining the concentrated sugar liquid may be returned as collected water to the saccharification tank T-1 and reused in the saccharification step (an arrow 15, ※1 in FIGURE).

### Fermentation Step

The fermentation step in the present embodiment is a step of adding yeast to the concentrated sugar liquid obtained in the step of obtaining the concentrated sugar liquid for fermentation to obtain hydrous bioethanol.

The concentrated sugar liquid obtained in the step of obtaining the concentrated sugar liquid is transferred to a fermentation tank T-5. In addition to the concentrated sugar liquid, yeast is also charged into the fermentation tank T-5. Steam, cooling water, a pH adjuster, and the like are optionally charged into the fermentation tank T-5.

In the fermentation step in the present embodiment, within the fermentation tank T-5, the concentrated sugar liquid is decomposed by yeast into ethanol and carbon dioxide (CO₂) under anaerobic conditions, and this reaction is termed fermentation.

The ethanol obtained above is referred to as hydrous bioethanol, with a concentration of around several tens of percent. For use as fuel, a purity close to 100% is required. Therefore, the hydrous bioethanol is optionally subjected to the subsequent concentration step after the unreacted yeast collection step.

### Unreacted Yeast Collection Step

The producing method according to the present embodiment preferably includes an unreacted yeast collection step after the fermentation step and before the concentration step.

In the unreacted yeast collection step in the present embodiment, the hydrous bioethanol in the fermentation tank T-5 is microfiltered through a ceramic membrane CM-2, so that unreacted yeast, which is yeast that has not been deactivated without reacting, can be separated and collected from the hydrous bioethanol, and the hydrous bioethanol is simultaneously purified.

It is preferable that the unreacted yeast collected as described above is returned as collected yeast to the fermentation tank T-5 again and subjected to the fermentation step (an arrow 21 in FIGURE).

By repeatedly using the yeast through collection and return of the unreacted yeast, an amount of yeast to be used can be reduced, leading to a reduction in production cost. In addition, reducing an amount of yeast contained in the waste liquid is advantageous from the viewpoint of reducing the environmental load. While repeatedly using the unreacted yeast, a new yeast may be charged into the fermentation tank T-5.

As the ceramic membrane CM-2 in the present embodiment, a ceramic membrane same as the ceramic membrane CM-1 in the enzyme collection step can be used, and a preferred aspect thereof is also the same.

That is, the ceramic membrane CM-2 preferably has filtration accuracy of 1.2 μm to 1.6 μm, for example.

In the producing method according to the present embodiment, the ceramic membrane CM-1 used in the enzyme collection step and the ceramic membrane CM-2 used in the unreacted yeast collection step may be the same as or different from each other.

Filtration using the above ceramic membrane CM-2 may be performed, for example, with a temperature of hydrous bioethanol at 30° C. to 40° C., a filtrate Flux at 80 (±5%) LMH, a circulation flow rate Flux at 25 times to 30 times (±5%) the LMH, and a circulation pressure at 0.6 MPa to 0.8 MPa (±5%). The present invention is not limited to these conditions.

Such stable production of hydrous bioethanol having a low impurity concentration by microfiltration leads to an increase in amount of bioethanol obtained in the subsequent concentration step, and also obtains an effect of reducing production cost from the viewpoint of implementing a high yield.

In the producing method according to the present embodiment, at least one of the enzyme collection step and the unreacted yeast collection step is preferably performed, both of them are more preferably performed, and as the enzyme collection step, a collection step using a ceramic membrane and a collection step using a UF membrane are further preferably performed.

### Concentration Step

The concentration step in the present embodiment is a step of concentrating the hydrous bioethanol obtained in the fermentation step to obtain bioethanol.

A method for concentrating hydrous bioethanol transferred to a fermentation liquid tank T-6 is not particularly limited, and a method known in the related art can be adopted. For example, the concentration is preferably performed by distillation, and the concentration is more preferably performed by vacuum distillation using a vacuum distillation device DE-1, preferably vacuum distillation.

The hydrous bioethanol transferred to the fermentation liquid tank T-6 undergoes the concentration step to become bioethanol, which is then transferred to a bioethanol tank T-7.

A concentration (a purity) of finally obtained bioethanol is preferably 99% or more, more preferably 99.3% or more, still more preferably 99.5% or more, with higher concentrations being more preferred.

Through the above steps, the production cost can be suitably reduced, and bioethanol can be obtained.

Water separated during the concentration for obtaining bioethanol may be returned as collected water (collected hot water) to the saccharification tank T-1 and reused in the saccharification step (※4 in FIGURE) .

### Protein-containing Waste Liquid Collection Step

The producing method according to the present embodiment may further include a step of collecting a waste liquid, and the waste liquid is preferably a protein-containing waste liquid.

In the producing method according to the present embodiment, the waste liquid is discharged at least after the saccharification step, the fermentation step, and the concentration step. The waste liquid after the saccharification step contains at least the enzyme and the sugar, the waste liquid after the fermentation step contains at least the yeast and the sugar, and the waste liquid after the concentration step contains the enzyme, the sugar, and the yeast that are not completely removed during the preceding steps. That is, the waste liquids after the above steps are all protein-containing waste liquids.

In the producing method according to the present embodiment, reusing the enzyme and the unreacted yeast through the enzyme collection step and the unreacted yeast collection step is one method for implementing cost reduction. However, as the enzyme is reused repeatedly, a function thereof gradually decreases, and there is a limit to the number of times the enzyme or unreacted yeast is reused, for example, the enzyme cannot be reused infinitely.

Therefore, in the saccharification step and the fermentation step, it is necessary to discharge the protein-containing waste liquid at a specific cycle.

Therefore, the producing method according to the present embodiment preferably further includes a step of collecting the protein-containing waste liquid after one or more steps of the saccharification step, the fermentation step, and the concentration step, more preferably further includes a step of collecting the protein-containing waste liquid after two or more steps thereof, and still more preferably further includes a step of collecting the protein-containing waste liquid after all of the three steps.

The above waste liquid contains proteins, so that when the waste liquid is subjected to a biological reaction, gas is generated. Specifically, when fermentation is performed under anaerobic conditions, biomass gas containing methane gas is obtained and can be used as renewable energy. The use of the renewable energy is not particularly limited, and for example, when the renewable energy is used for power supply driving for producing bioethanol according to the present embodiment, it can also contribute to a reduction in production cost of bioethanol.

Therefore, the producing method according to the present embodiment more preferably further includes a step of producing and collecting methane gas using the collected protein-containing waste liquid.

The above does not exclude generation of other gases together with the methane gas. That is, the biomass gas containing methane gas may be produced and collected using the protein-containing waste liquid, and the biomass gas may contain carbon dioxide and the like in addition to methane gas.

In the step of producing and collecting methane gas in the present embodiment, the protein-containing waste liquid discharged through the saccharification step performed in the saccharification tank T-1 is transferred into an anaerobic methane gas fermentation device MFE-1 (an arrow ※2 in FIGURE), the protein-containing waste liquid discharged through the fermentation step performed in the fermentation tank T-5 is transferred into the anaerobic methane gas fermentation device MFE-1 (an arrow ※3 in FIGURE), and the protein-containing waste liquid discharged when concentrating the hydrous bioethanol in the fermentation liquid tank T-6 is transferred into the anaerobic methane gas fermentation device MFE-1 (an arrow ※5 in FIGURE). Then, methane gas is produced by mixing and reacting with methane fermentation bacteria in the anaerobic methane gas fermentation device MFE-1.

By collecting the produced methane gas and purifying methane gas, power can be generated by a gas turbine. The generated electricity can be all reused as a power supply necessary for operation energy and operation management of a device for producing bioethanol according to the present embodiment.

### Device for Producing Bioethanol

The device for producing bioethanol according to the present embodiment is used when performing the producing method described in <<Method for Producing Bioethanol>> above.

An aspect of the device for producing bioethanol according to the present embodiment includes the saccharification tank T-1, the sugar liquid tank T-2, the fermentation tank T-5, the bioethanol tank T-7, and the ceramic membrane.

Here, the saccharification tank T-1 is a tank to be further added with an enzyme to charged cellulose for saccharification to obtain a sugar liquid.

The sugar liquid tank T-2 is a tank to be supplied with a concentrated sugar liquid obtained by concentrating the sugar liquid generated in the saccharification tank T-1.

The fermentation tank T-5 is a tank to be further added with yeast to the charged concentrated sugar liquid for fermentation to obtain hydrous bioethanol.

The bioethanol tank T-7 is a tank to be supplied with bioethanol obtained by concentrating the hydrous bioethanol generated in the fermentation tank T-5.

The ceramic membrane is preferably provided in at least one of between the sugar liquid tank T-2 and the fermentation tank T-5 and between the fermentation tank T-5 and the bioethanol tank T-7, and more preferably provided in both.

The enzyme is separated from the sugar liquid contained in the sugar liquid tank T-2 by the ceramic membrane CM-1 provided between the sugar liquid tank T-2 and the fermentation tank T-5. In addition to the enzyme, the undecomposed cellulose can also be separated, and the sugar liquid is purified.

The unreacted yeast is separated from the hydrous bioethanol contained in the fermentation tank T-5 by the ceramic membrane CM-2 provided between the fermentation tank T-5 and the bioethanol tank T-7. The hydrous bioethanol is purified.

When the ceramic membrane CM-2 is provided between the fermentation tank T-5 and the bioethanol tank T-7, the hydrous bioethanol having passed through the ceramic membrane CM-2 is transferred to the fermentation liquid tank T-6.

Another aspect of the device for producing bioethanol according to the present embodiment includes the saccharification tank T-1, the sugar liquid tank T-2, the fermentation tank T-5, the bioethanol tank T-7, and the protein-supported polyamide RO membrane.

Here, the saccharification tank T-1, the sugar liquid tank T-2, the fermentation tank T-5, and the bioethanol tank T-7 are the same as those in the above-described aspect.

The protein-supported polyamide RO membrane is provided between the sugar liquid tank and the fermentation tank T-5. Here, when the producing device according to the present embodiment does not include the ceramic membrane CM-1 and the UF membrane UF-1, the sugar liquid tanks T-3 and T-4 are not necessary, and the sugar liquid in the sugar liquid tank T-2 is subjected to the filtration by the protein-supported polyamide RO membrane. When the producing device according to the present embodiment does not include the UF membrane UF-1, the sugar liquid tank T-4 is not necessary, and the sugar liquid in the sugar liquid tank T-3 is subjected to the filtration by the protein-supported polyamide RO membrane.

The device for producing bioethanol according to the present embodiment may include each constituent component described in <<Method for Producing Bioethanol>> above.

Examples thereof include the anaerobic methane gas fermentation device MFE-1 for charging a protein-containing waste liquid (an enzyme-containing waste liquid) from the saccharification tank T-1, a protein-containing waste liquid (a yeast-containing waste liquid) from the fermentation tank T-5, and a protein-containing waste liquid (a distilled waste liquid) from the fermentation liquid tank T-6, a device for purifying methane gas and generating power, a bioethanol producing device power supply that can be driven using produced methane gas, a system for supplying a collected enzyme collected from the sugar liquid to the liquefaction tank T-1 (arrows 9 and 12 in FIGURE), a system for supplying an unreacted yeast collected from hydrous bioethanol to the fermentation tank T-5 (an arrow 21 in FIGURE), a system for supplying, as collected water, water obtained when concentrating the sugar liquid into a concentrated sugar liquid to the saccharification tank T-1 (※1 in FIGURE) , and a system for supplying, as collected hot water, hot water obtained when distilling hydrous bioethanol to the saccharification tank T-1 (※4 in FIGURE) . Pipes for connecting these configurations and pumps (P-1 to P-7) are also included.

In addition to the above, a system for discharging each of the protein-containing waste liquid (the enzyme-containing waste liquid) from the saccharification tank T-1, the protein-containing waste liquid (the yeast-containing waste liquid) from the fermentation tank T-5, and the protein-containing waste liquid (the distilled waste liquid) from the fermentation liquid tank T-6 in a predetermined batch number may be further provided.

### Example

Hereinafter, the present invention will be described with reference to a test example, but the present invention is not limited thereto.

A sugar liquid was obtained by charging sheet pulp as raw material pulp containing cellulose, industrial water, and an enzyme (cellulase), caustic soda as a pH adjuster, and a fatty acid-based defoamer as a defoamer into the saccharification tank T-1, maintaining a temperature in the tank at 50° C. to 55° C., and saccharifying the cellulose. At this time, since a collected enzyme to be described later is accumulated in the saccharification tank T-1 at 80% with respect to the active FPU 100% addition amount, the enzyme (the cellulase) was supplied only in an insufficient amount of enzyme activity FPU 20%.

Generally, in the above reaction, since it is necessary to add an enzyme activity of 30 FPU/g-pulp per kg of a raw material having a solid content concentration of 54%, it is necessary to add 162 g of a new enzyme for each saccharification step. On the other hand, in the saccharification step in the producing method according to the present embodiment, since a saccharification rate of 70% to 80% or more is maintained and the collected enzyme can be continuously reused in five batches or more, an amount of enzyme to be newly added is 20% (33 g), and a sugar liquid (Brix value of 8% to 9%) can be produced with a significantly small consumption amount.

Accordingly, a mixed liquid of the sugar liquid 80% and the undecomposed cellulose 20% was obtained. The undecomposed cellulose had a particle size of 4μm to 1,000 μm and was contained in the mixed liquid.

Next, the mixed liquid was transferred to the sugar liquid tank T-2 and subjected to cross-flow treatment with a ceramic membrane (manufactured by TAMI) having filtration accuracy of 1.4 μm, thereby performing microfiltration for separating the undecomposed cellulose and the sugar liquid and simultaneously collecting the enzyme incorporated into a substrate.

The cross-flow treatment was performed under conditions of a liquid temperature of the mixed liquid: 40° C. to 50° C., a filtrate Flux: 70 (±10%) LMH, a circulation flow rate Flux: 30 (±5%) times, and a circulation pressure: 0.6 MPa (±5%).

As a result, a sugar liquid having a Brix value of 9% to 10% was obtained in the sugar liquid tank T-3.

The collected enzyme was again charged into the saccharification tank T-1.

Subsequently, the sugar liquid having a Brix value of 9% to 10% was treated with a UF membrane (manufactured by Kuraray Co., Ltd.) having a molecular weight cut-off of MW-5000 to collect a smaller enzyme incorporated into the substrate.

The above treatment was performed under conditions of a liquid temperature of the mixed liquid: 30° C. to 50° C., a Flux: 80 (±10%) LMH, a circulation flow rate Flux: 10 to 15 (±5%) times, and a circulation pressure: 0.5 MPa (±5%).

As a result, a sugar liquid having a Brix value of 9.5% to 10.5% was obtained in the sugar liquid tank T-4.

The collected enzyme was again charged into the saccharification tank T-1.

The purified sugar liquid having a Brix value of 9.5% to 10.5% was separated into desalted water and a concentrated sugar liquid having a Brix value of 20% or more using a protein-supported polyamide RO membrane (manufactured by Aquaporin). The concentrated sugar liquid having a Brix value of 20% or more was transferred to the fermentation tank T-5.

Here, the separation was performed under conditions of an operation pressure: 3.7 (+3) MPa and a Flux: 9 LMH, and a desalination rate was 99.5% or more.

The power consumption required to obtain 1 L of the concentrated sugar liquid having a Brix value of 20% or more was 0.01 kW, and it was confirmed that the concentrated sugar liquid was obtained with low energy as compared with the power consumption required in the related art (for reference, the power consumption required when an evaporation device (MVR type) or a high-pressure reverse osmosis membrane was used was about 0.04 kW or more).

Water separately obtained when obtaining the concentrated sugar liquid was charged as collected water into the saccharification tank T-1.

Next, yeast, steam, cooling water, and caustic soda as a pH adjuster were charged into the fermentation tank T-5 containing the concentrated sugar liquid, and the concentrated sugar liquid was fermented while maintaining the temperature in the tank at 30° C. to 35° C. to obtain hydrous bioethanol.

Here, for an amount of yeast to be added, the amount of yeast to be added was 70% of the insufficient amount, since about 30% activity of collected yeast to be described later was deposited per batch in the fermentation tank T-5. As described above, in the fermentation step in the producing method according to the present embodiment, an amount of yeast to be newly added can be reduced. The amount of yeast to be newly added can be reduced since the collected yeast can be reused a plurality of times until it is deactivated.

The hydrous bioethanol in the fermentation tank T-5 obtained above was subjected to cross-flow treatment with a ceramic membrane (manufactured by TAMI) having filtration accuracy of 1.4 μm to perform microfiltration, and the unreacted yeast taken into the substrate was also simultaneously collected.

The cross-flow treatment was performed under conditions of a liquid temperature of the hydrous bioethanol: 30° C. to 40° C., a filtrate Flux: 80 LMH, a circulation flow rate Flux: 30 times, and a circulation pressure: 0.6 MPa to 0.8 MPa.

As a result, 8 vol % to 9 vol % of hydrous bioethanol was obtained in the fermentation liquid tank T-6.

The collected unreacted yeast was charged again into the fermentation tank T-5.

The above-obtained hydrous bioethanol in the fermentation liquid tank T-6 was distilled using a vacuum distillation device DE-1 under a condition of a distillation time of 3 hours to produce bioethanol.

The distillation was performed under a condition of a distillation temperature of 78° C. to 82° C.

Hot water obtained during the distillation is used as hot water as a heating medium in a temperature raising step of the saccharification tank T-1.

The collected enzyme and the collected yeast were discarded after five batches, but the protein-containing waste liquid discharged in the saccharification step, the fermentation step, and the concentration step contained a large amount of protein, an amount of biogas generated per wet weight was 174 NL/kg, and an amount of gas generated per organic substance was 2,063 NL/kg-VS. A very high level of biogas can be collected from the protein-containing waste liquid.

This is because it is said that an upper limit of an ammoniacal nitrogen concentration in a tank in medium-temperature fermentation (fermentation temperature of about 37° C.) of a wet method is generally 3,000 mg/L to 4,000 mg/L, and when adopting the producing method according to the present embodiment, the ammoniacal nitrogen concentration is estimated to be about 1,700 mg/L, and it can be confirmed that the operation is not hindered.

A methane concentration in the biogas obtained by fermenting the obtained protein-containing waste liquid was 57%, and methane gas with a high concentration and a methane generation rate of 98.9 L/kg was collected.

Then, the power generation amount obtained when 1 kg of the protein-containing waste liquid was treated was 0.314 kWh, but the power amount of the renewable energy can also cover 65% to 70% of the operation energy of the bioethanol producing device according to the present embodiment.

As described above, according to the producing method of the present embodiment, amounts of enzymes and yeasts to be used can be reduced, and concentration and the like can be efficiently performed with low energy. In addition to the above, the protein-containing waste liquid discharged from the production process can be independently and effectively used for energy related to the production of bioethanol and the operation of the producing device.

### REFERENCE SIGNS LIST

T-1: saccharification tank
T-2, T-3, T-4: sugar liquid tank
T-5: fermentation tank
T-6: fermentation liquid tank
T-7: bioethanol tank
P-1: saccharification pump
P-2: ceramic membrane pump
P-3: UF membrane pump
P-4: concentration pump
P-5: concentrated sugar liquid pump
P-6: fermentation pump
P-7: fermentation liquid pump
CM-1, CM-2: ceramic membrane
UF-1: UF membrane
RO-1: RO membrane
DE-1: vacuum distillation device
MFE-1: anaerobic methane gas fermentation device

1. A method for producing bioethanol, which is a method for producing bioethanol from cellulose, the method comprising: in order,
a saccharification step of adding an enzyme to the cellulose for saccharification to obtain a sugar liquid;
a step of obtaining a concentrated sugar liquid by concentrating the obtained sugar liquid;
a fermentation step of adding yeast to the concentrated sugar liquid for fermentation to obtain hydrous bioethanol; and
a concentration step of concentrating the hydrous bioethanol to obtain bioethanol, wherein
the method further includes at least one step of an enzyme collection step performed after the saccharification step and an unreacted yeast collection step performed after the fermentation step,
the enzyme collection step includes a step of collecting the enzyme using a ceramic membrane, and
in the unreacted yeast collection step, a ceramic membrane is used.

2. A method for producing bioethanol, which is a method for producing bioethanol from cellulose, the method comprising: in order,
a saccharification step of adding an enzyme to the cellulose for saccharification to obtain a sugar liquid;
a step of obtaining a concentrated sugar liquid by concentrating the obtained sugar liquid;
a fermentation step of adding yeast to the concentrated sugar liquid for fermentation to obtain hydrous bioethanol; and
a concentration step of concentrating the hydrous bioethanol to obtain bioethanol, wherein
in the step of obtaining the concentrated sugar liquid, the sugar liquid is concentrated using a protein-supported polyamide RO membrane.

3. The method for producing bioethanol according to claim 1, wherein
the enzyme collection step further includes a step of collecting the enzyme using a UF membrane after the step of collecting the enzyme using the ceramic membrane.

4. The method for producing bioethanol according to claim 3, wherein
a UF membrane having a molecular weight cut-off of MW-3000 to MW-7000 is used as the UF membrane.

5. The method for producing bioethanol according to claim 1, wherein
in the enzyme collection step, undecomposed cellulose is also collected together with the enzyme.

6. The method for producing bioethanol according to claim 1, wherein
the enzyme collection step and the unreacted yeast collection step are both performed.

7. The method for producing bioethanol according to claim 1, wherein
in the step of obtaining the concentrated sugar liquid, the sugar liquid is concentrated using an RO membrane.

8. The method for producing bioethanol according to claim 7, wherein
a protein-supported polyamide RO membrane is used as the RO membrane.

9. The method for producing bioethanol according to claim 1, wherein
in at least one of the enzyme collection step and the unreacted yeast collection step, a ceramic membrane having filtration accuracy of 1.2 μm to 1.6 μm is used as the ceramic membrane.

10. The method for producing bioethanol according to claim 1, wherein
in the concentration step, the hydrous bioethanol is concentrated by distillation.

11. The method for producing bioethanol according to claim 1, further comprising:
a step of collecting a protein-containing waste liquid after one or more steps of the saccharification step, the fermentation step, and the concentration step.

12. The method for producing bioethanol according to claim 11, further comprising:
a step of producing and collecting methane gas using the collected protein-containing waste liquid.

13. The method for producing bioethanol according to claim 2, wherein
in the concentration step, the hydrous bioethanol is concentrated by distillation.

14. The method for producing bioethanol according to claim 2, further comprising:
a step of collecting a protein-containing waste liquid after one or more steps of the saccharification step, the fermentation step, and the concentration step.

15. The method for producing bioethanol according to claim 14, further comprising:
a step of producing and collecting methane gas using the collected protein-containing waste liquid.

16. A device for producing bioethanol, which is a device for producing bioethanol from cellulose, the device comprising:
a saccharification tank configured to allow an enzyme to be added to the cellulose for saccharification to obtain a sugar liquid;
a sugar liquid tank configured to allow a concentrated sugar liquid obtained by concentrating the obtained sugar liquid to be supplied;
a fermentation tank configured to allow yeast to be added to the concentrated sugar liquid for fermentation to obtain hydrous bioethanol; and
a bioethanol tank configured to allow bioethanol obtained by concentrating the hydrous bioethanol to be supplied, wherein
at least one of a ceramic membrane for separating the enzyme that is provided between the sugar liquid tank and the fermentation tank and a ceramic membrane for separating unreacted yeast that is provided between the fermentation tank and the bioethanol tank is further provided.

17. A device for producing bioethanol, which is a device for producing bioethanol from cellulose, the device comprising:
a saccharification tank configured to allow an enzyme to be added to the cellulose for saccharification to obtain a sugar liquid;
a sugar liquid tank configured to allow a concentrated sugar liquid obtained by concentrating the obtained sugar liquid to be supplied;
a fermentation tank configured to allow yeast to be added to the concentrated sugar liquid for fermentation to obtain hydrous bioethanol; and
a bioethanol tank configured to allow bioethanol obtained by concentrating the hydrous bioethanol to be supplied, wherein
a protein-supported polyamide RO membrane for concentrating the sugar liquid is provided between the sugar liquid tank and the fermentation tank.

18. The device for producing bioethanol according to claim 16, wherein
a protein-supported polyamide RO membrane for concentrating the sugar liquid is provided between the sugar liquid tank and the fermentation tank.
